(19) United States
(12) Patent Application Publication
(10) Pub. No.: US 2018/0185111 A1
(43) Pub. Date: Jul. 5, 2018

(54) BIOPSY SYSTEM AND BIOPSY DEVICE HOLDER FOR THE SAME

(71) Applicant: Devicor Medical Products, Inc., Cincinnati, OH (US)

(72) Inventors: David Shuart, Mason, OH (US); Jordan Rebellino, Cincinnati, OH (US); Andrew Robinson, Cincinnati, OH (US); Jodi Hafemeister, Liberty Township, OH (US); Gary Wagner, Independence, OH (US); Rachel Choung, Cincinnati, OH (US); Emmanuel Tanghal, Mason, OH (US); Umashankar Chockalingam, Loveland, OH (US)

(21) Appl. No.: 15/855,207

(22) Filed: Dec. 27, 2017

Related U.S. Application Data
(60) Provisional application No. 62/440,162, filed on Dec. 29, 2016.

(51) Int. Cl. A61B 50/13; B62B 3/04

(52) U.S. Cl. CPC A61B 50/13; A61B 10/0233; B62B 3/04

(57) ABSTRACT

A biopsy system includes a cart and a biopsy device holder. The biopsy device holder is coupled to the cart. The biopsy device holder includes a front holder portion and a rear holder portion. Each holder portion defines an upper support and a lower support. The upper support is configured to support a first biopsy device in lieu of the second biopsy device. The lower support is configured to support a second biopsy device in lieu of the first biopsy device.

### PRIORITY

This present application claims priority to U.S. Provisional Patent Application No. 62/440,162, entitled “Ubiquitous Device Holder for Mobile Medical Cart,” filed on Dec. 29, 2016, the disclosure of which is hereby incorporated by reference herein in its entirety.

### BACKGROUND

The state of the art for breast biopsy is vacuum-assisted breast biopsy. A current textbook in this area is “Vacuum-Assisted Breast Biopsy with Mammotome®, written by Markus Hahn, Anne Tardivon and Jan Casselman, available Nov. 11, 2012, copyright 2013 by Devicor Medical Germany GmBh, published in Germany by Springer Medizin Verlag, ISBN 978-3-642-34270-7.

Biopsy devices may be used under ultrasound image guidance, stereotactic (X-ray) guidance, MRI guidance, Positron Emission Mammography (“PEM” guidance), Breast Specific Gamma Imaging (“BSGI”) guidance, or other guidance. Each procedure has its own methodology based on the type of imaging guidance used. The following briefly describes ultrasound image guided biopsy procedures, stereotactic guided biopsy procedures and MRI guided biopsy procedures.

In an ultrasound image guided breast biopsy procedure, the operator may position an ultrasound transducer on the patient's breast and maneuver the transducer while viewing an ultrasound image display screen to locate suspicious tissue in the patient's breast. Once the operator locates the suspicious tissue, the operator may anesthetize the target region of the breast. Once the breast has been anesthetized, the operator may create an initial incision using a scalpel at a location on the exterior of the breast offset from the transducer. A needle of a breast biopsy probe disposed coaxially within an introducer cannula is then inserted into the breast through the initial incision. The operator continues to hold the ultrasound transducer with one hand while maneuvering the biopsy probe with the other hand. While viewing the ultrasound image on the display screen, the operator guides the needle to a position adjacent to the suspicious tissue. A cutter within the needle of the probe is used to remove tissue which is then conveyed either to a manual pick-up location on the breast biopsy device or to a tissue sample chamber. The needle of the breast biopsy device is then removed, leaving the introducer cannula disposed within the breast. The introducer cannula may then be used to introduce a biopsy marker cannula for deploying a biopsy site marker at the biopsy site. Once a marker has been deployed at the biopsy site, the biopsy marker cannula and the introducer cannula are both removed from the breast and the incision is closed using a medically acceptable way to close breaks in the skin.

In a stereotactic image guided breast biopsy procedure, the patient is first positioned relative to x-ray equipment, which includes a breast localization assembly. In some procedures, the patient is oriented in a prone position, with the patient lying face down on a procedure table with at least one breast hanging pendulously through an aperture in the procedure table. The breast is then compressed between a compression paddle and an x-ray receptor of a localization assembly that is positioned under the procedure table. A breast biopsy device is positioned on an automatic guide device in front of the compression paddle and between the breast and an x-ray source. Once positioning of the patient and localization of the breast are complete, a scout image is acquired with the x-ray receptor in a zero-degree angular position (i.e., the x-rays are emitted along an axis normal relative to the x-ray receptor). If the scout image indicates that the patient has been positioned in a desired position, the procedure may proceed with the acquisition of stereotactic image pairs. Stereotactic image pairs are acquired by orienting the x-ray source at various complementary angular positions relative to the x-ray receptor (e.g., +15° and −15°), with at least one x-ray image acquired at each position.

Further in the stereotactic image guided breast biopsy procedure, once a suitable stereotactic image pair is acquired, an operator may identify a target site where biopsy sampling is desired by examining the stereotactic image pair. The target site is marked on each stereotactic image and a precise location of the target site on a Cartesian coordinate system is computed using an image processing module. The computed location of the target site is then communicated to the automatic guide device. The automatic guide device is responsive to this information to position the breast biopsy probe into a position that aligns with the target site. With the breast biopsy device positioned, an operator may then fire a needle of the biopsy probe into the breast of the patient, thereby positioning the needle at the target site. A cutter within the needle of the prove is then used to remove tissue, which is then conveyed either to a manual pick-up location on the breast biopsy device or to a tissue sample chamber. After the biopsy tissue is removed, a biopsy marker cannula is inserted into the needle and is used to deploy a biopsy site marker at the biopsy site. Once a marker has been deployed at the biopsy site, the needle is removed from the breast and the incision is closed using a medically acceptable way to close breaks in the skin.

In an MRI guided breast biopsy procedure, after the patient is properly positioned on the table and a targeting device (e.g., a grid and cube combination or a pillar, post and cradle support combination) has been deployed and used, a baseline MRI image is taken to verify the target location. After that, a scalpel is used to incise the skin of the breast. Next, an assembly, formed by an obturator disposed in a sleeve, is inserted through the incision to penetrate the breast tissue under the skin. In some acceptable surgical techniques, the obturator is removed and an imaging rod is inserted into the sleeve in place of the obturator. An imaging rod is defined simply as an appropriately shaped rod that includes a feature that is detectable by an imaging technique being used for the biopsy procedure. The MRI image of the imaging rod is used to locate the site to which the sleeve/obturator assembly has penetrated. In some other acceptable surgical techniques, the obturator cooperates with the breast tissue to provide a visually observable artifact in an MRI image. With both techniques, after the location within the breast where the biopsy is to be taken is confirmed, the obturator or the imaging rod is removed.

Further in the MRI guided breast biopsy procedure, after the obturator or imaging rod has been removed, it is replaced in the sleeve with the needle of a breast biopsy probe. A cutter within the needle of the probe is used to remove tissue, which is then conveyed either to a manual pick up location on the breast biopsy device or to a breast biopsy device sample chamber. After the biopsy tissue is removed, a biopsy marker cannula is inserted into the needle and is used to deploy a biopsy site marker at the biopsy site. The needle is then removed from the sleeve. Optionally, the imaging rod or the obturator is put back into the breast for reimaging of the biopsy site. Subsequently, the imaging rod or obturator and the sleeve are removed.

In a medical facility, a biopsy device and related equipment typically need to be moved between various locations such as storage areas, procedure rooms and even operating rooms. Various carts are available for moving the biopsy device and related equipment between locations. Such carts may carry, for example, a biopsy probe, holster, tube set, vacuum source, control module, X-Ray cabinet, medical grade monitor, CPU, keyboard, mouse, fluid canister, foot pedal, specimen radiograph unit, and keypad, among other features/devices. Current carts often require specialized holders for medical devices to secure them safely to the cart. There is a need for an improved cart with improved functionality.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of a conventional cart for transporting a biopsy device and related equipment;

FIG. 2 depicts a perspective view of an exemplary biopsy device attached to a biopsy device holder of the cart of FIG. 1;

FIG. 3 depicts a perspective view of another exemplary biopsy device attached to the biopsy device holder of FIG. 2;

FIG. 4 depicts a perspective view of an exemplary cart;

FIG. 5 depicts a top plan view of the cart of FIG. 4;

FIG. 6 depicts a front elevational view of the cart of FIG. 4;

FIG. 7 depicts side elevational view of an exemplary biopsy device disposed within an exemplary biopsy device holder of the cart of FIG. 4;

FIG. 8 depicts a perspective view of a front cradle of the biopsy device holder of FIG. 7;

FIG. 9 depicts a rear elevational view of the front cradle of FIG. 8;

FIG. 10 depicts a side elevational view of the front cradle of FIG. 8;

FIG. 11 depicts a top plan view of the front cradle of FIG. 8;

FIG. 12 depicts a perspective view of a rear cradle of the biopsy device holder of FIG. 7;

FIG. 13 depicts a front elevational view of the rear cradle of FIG. 12;

FIG. 14 depicts a side elevational view of the rear cradle of FIG. 12;

FIG. 15 depicts a top plan view of the rear cradle of FIG. 12;

FIG. 16 depicts a perspective view of the biopsy device of FIG. 7 disposed in another exemplary biopsy device holder;

FIG. 17 depicts another perspective view of the biopsy device of FIG. 7 disposed in the biopsy device holder of FIG. 16;

FIG. 18 depicts a perspective view of the biopsy device holder of FIG. 16;

FIG. 19 depicts a detailed perspective view of another biopsy device engaging a portion of the biopsy device holder of FIG. 16;

FIG. 20 depicts another detailed perspective view of the biopsy device of FIG. 19 engaging the portion of the biopsy device holder of FIG. 19;

FIG. 21 depicts detailed rear elevational view of another biopsy device engaging another biopsy device holder;

FIG. 22 depicts a perspective view another exemplary biopsy device engaging still another exemplary biopsy device holder;

FIG. 23 depicts a perspective view of another exemplary biopsy device engaging the biopsy device holder of FIG. 22;

FIG. 24 depicts a perspective view of a front cradle of the biopsy device holder of FIG. 22;

FIG. 25 depicts a rear elevational view of the front cradle of FIG. 24;

FIG. 26 depicts a side elevational view of the front cradle of FIG. 24;

FIG. 27 depicts a top plan view of the front cradle of FIG. 24;

FIG. 28 depicts a perspective view of a rear cradle of the biopsy device holder of FIG. 22;

FIG. 29 depicts a front elevational view of the rear cradle of FIG. 28;

FIG. 30 depicts a side elevational view of the rear cradle of FIG. 28; and

FIG. 31 depicts a top plan view of the rear cradle of FIG. 28.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

Generally, the present disclosure provides for an improved cart for transporting a biopsy device. The cart may include a biopsy device holder that may support multiple different types of biopsy devices. For example, the biopsy device holder may include at least two pairs of upwardly extending arms. The upwardly extending arms may conform to at least a portion of a bottom surface of each different biopsy device. The arms may be flexible and resilient to accommodate different designs and retain the biopsy device between the arms. The biopsy device may be positioned horizontally level to prevent exposure of a sharp end. The cart may include a rail having a notch for retaining and protecting the sharp end of the biopsy device. The cart may also carry biopsy related equipment such as a specimen radiograph. The cart may provide targeted lighting for various surfaces of the cart such as the surface of a collapsible tray and the interior of a retractable sample tray.

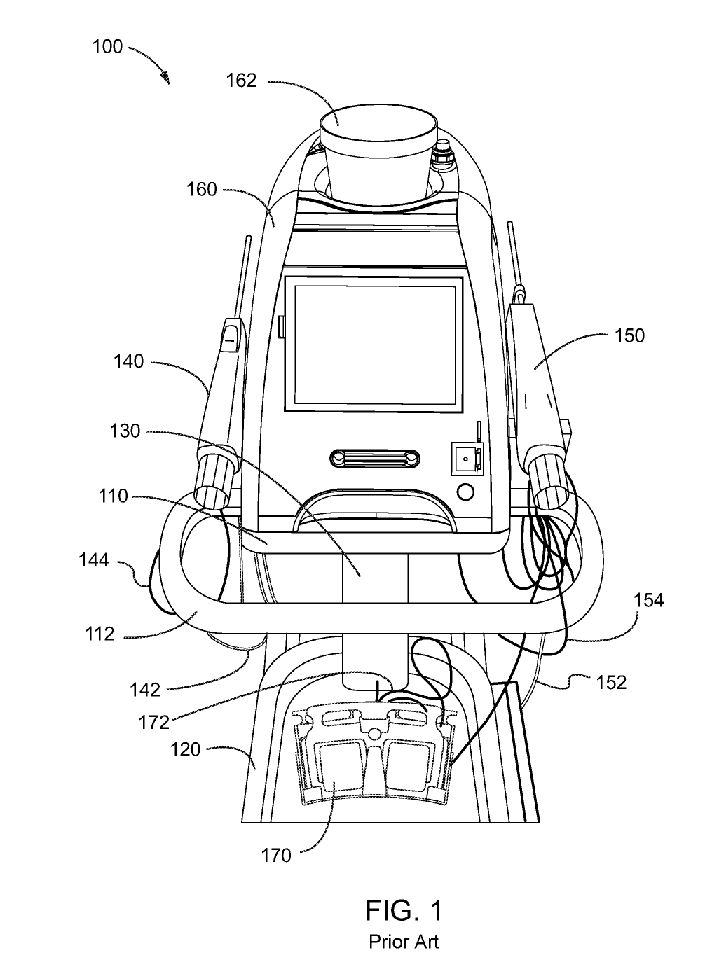

FIG. 1 illustrates a prior art cart 100 for transporting a biopsy device and related equipment. The cart may include an upper shelf 110 and a lower shelf 120 connected via a column 130. The upper shelf 110 may support, for example, a first biopsy device 140, a second biopsy device 150, and a control unit 160. A handle 112 may be attached to the upper shelf 110 at the front of the cart 100. The first biopsy device 140 may use a first modality (e.g., ultrasound). The second biopsy device 150 may use a second modality (e.g., stereotactic x-ray). The first biopsy device 140 and the second biopsy device 150 may be coupled to the control unit 160 via tubing 142, 152 and wires 144, 154. A collection canister 162 may be stored on top of the control unit 160, or otherwise supported on the cart 100. The lower shelf 120 may support foot controls 170. The foot controls 170 may be connected to the control unit 160 via wires 172.

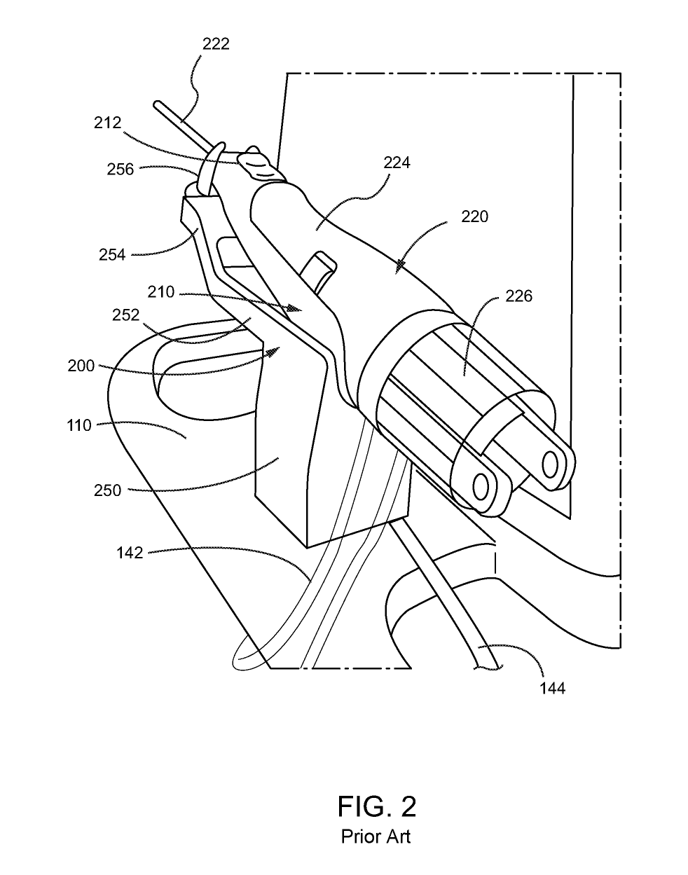

FIG. 2 illustrates the example biopsy device 140 and prior art biopsy device holder 200. The biopsy device 140 may be used, for example, with an ultrasound modality. Generally, the biopsy device 140 may include a reusable holster 210 and a disposable probe 220. The reusable holster 210 includes a bottom surface that can contact the biopsy device holder 200. Internally, the reusable holster 210 can include hardware (e.g., motors, gears, switches) (not shown) for operating the biopsy device 140. The reusable holster 210 also includes controls 212 located at a distal end of the reusable holster. The disposable probe 220 may include parts that come into contact with a patient and/or bodily fluids. For example, the disposable probe 220 may include a needle 222, a body 224, and a sample container 226.

The biopsy device holder 200 may include a base 250, arms 252, distal support 254, and a stop 256. The base 250 may be attached to the upper shelf 110 and extend upward. The base 250 may have a curved cutout to receive the bottom surface of the reusable holster 210. The sample container 226 may extend forward beyond the edge of the base 250. The tubing 142 may extend downward from the sample container 226. The wires 144 may extend downward from the holster 210 and through a slot in the base 250. The arms 252 extend rearward at an angle toward the distal support 254. The distal support 254 may connect the arms 252 and may include a curved cutout for receiving the bottom surface of the holster at a distal end. The stop 256 may extend rearward of the distal support 254 and may include a pair of arms. The needle 222 may extend between the arms of the stop 256. The distal end of the holster 210 may contact the forward surface of the stop.

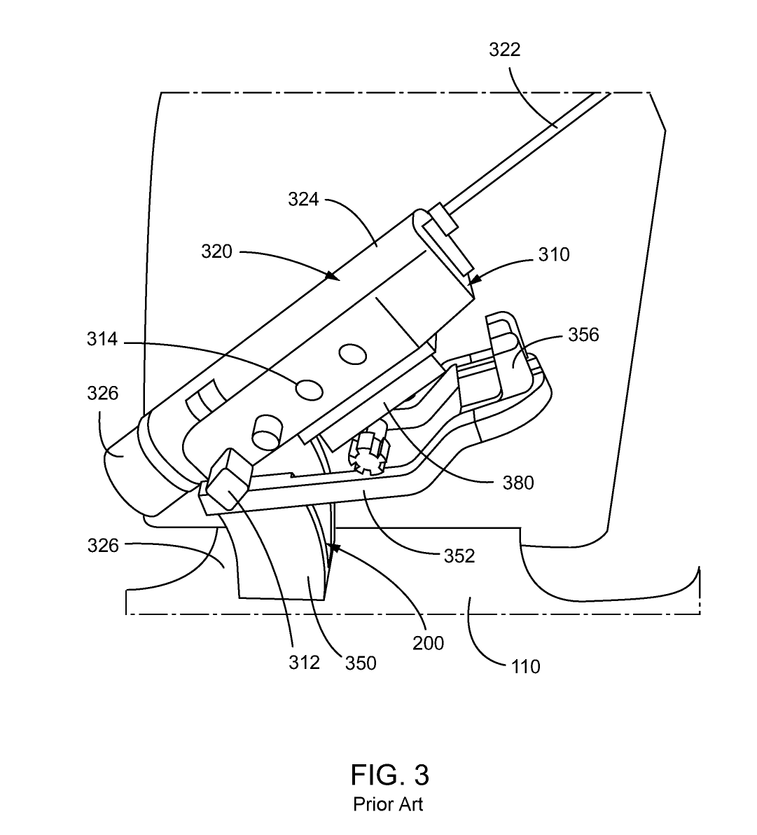

FIG. 3 illustrates another example biopsy device 150 and prior art biopsy device holder 300. In this case, the example biopsy device 150 may be used in a stereotactic modality. Similar to the example biopsy device 140, the example biopsy device 150 may also include a reusable holster 310 and a disposable probe 320. The reusable holster 310 may include a bottom surface that may contact the biopsy device holder 300. Internally, the reusable holster 310 can include hardware (e.g., motors, gears, switches) (not shown) for operating the biopsy device 140. The reusable holster 310 also includes controls 314 located on a side of the reusable holster 310. The disposable probe 320 may include parts that come into contact with a patient and/or bodily fluids. For example, the disposable probe 320 can include a needle 322, a body 324, and a collection container 326.

The biopsy device holder 300 may include a base 350, arms 352, distal support 354, and a stop 356. It should be noted that although the biopsy device holder 300 includes similar parts to the biopsy device holder 200, the biopsy device holder 300 is different from the biopsy device holder 200 to accommodate the biopsy device 150. The base 350 may be attached to the upper shelf 110 and extend upward. The base 350 may have two upwardly extending sides 351. The sides 351 may have a flat top surface. A proximal end of the holster 310 may rest on the flat surface. The collection container 326 may extend forward beyond the edge of the base 350. The tubing 152 may extend downward from the collection container 326. The wires 154 may extend proximally from the holster 210 between the sides 351 toward the front of the cart 100. The arms 352 may extend rearward from the sides 351 at an angle toward the distal support 354. The distal support 354 may connect the arms 352 and may support the bottom surface of the holster 310 at a distal end. The stop 356 may include a pair of arms extending upward from the distal support 354. The needle 322 may extend between the arms of the stop 356. In an aspect, the distal end of the holster 310 may contact the forward surface of the stop.

In the illustrated example, the biopsy device 150 may further include an adaptor 380.

The biopsy device 150 may be used in a variety of medical settings. For example, different offices or operating rooms may have different procedure tables for positioning a patient. The biopsy device 150 may be positioned using stereotactic images of the breast. The adaptor 380 may be used to attach the biopsy device 150 to a procedure table or automatic guide device for proper positioning. For example, the adaptor 380 may be mounted to the bottom surface of the holster 310. The adaptor 380, however, may not be designed for use with the biopsy device holder 300. For example, as illustrated, the adaptor 380 may contact the arms 352 of the biopsy device holder 300 and support the biopsy device 150 on the arms 352 rather than on the distal support 354. This may increase the angle of the needle 322. Some adaptors may increase the angle of the biopsy device 150 to such a degree that the sample container 226 may contact the upper shelf 110. Further, the biopsy device 150 may be able to inadvertently move because the biopsy device 150 may be insufficiently restrained. For example, the needle 322 may not be positioned between the arms of the stop 356.

In view of the above, although the cart 100 with the prior art biopsy device holders 200, 300 has proven suitable for transporting a biopsy device in a medical facility, several drawbacks have been observed. For example, although the control unit 160 may be used with multiple biopsy devices, two different biopsy device holders 200 and 300 may be used to mount the biopsy device. Typically, only one biopsy device may be located on the cart 100 and the additional space that may be used by the second biopsy device holder is wasted. Additionally, the needle 222, 322 may be angled upward. The needle 222, 322 may be razor sharp. Although the needle 222, 322 may be covered with a protective sleeve during transportation, it is possible to remove the sleeve and expose the needle 222. Accordingly, there is a risk of injury associated with the location and orientation of the needle 222, 322. In another aspect, the biopsy device 140, 150 may come into contact with bodily fluids during a procedure and retain such fluids after a procedure. The upward angle of the biopsy device 140, 150 may result in retained fluids flowing out of the biopsy device 140 during transport. In view of the above, the present disclosure addresses these drawbacks of a conventional cart.

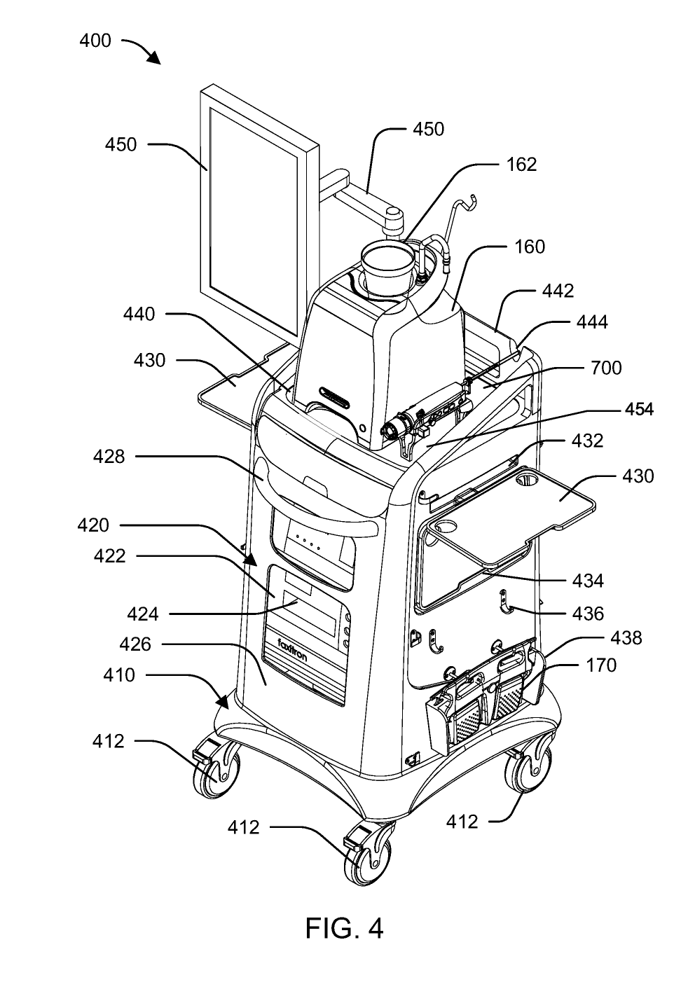

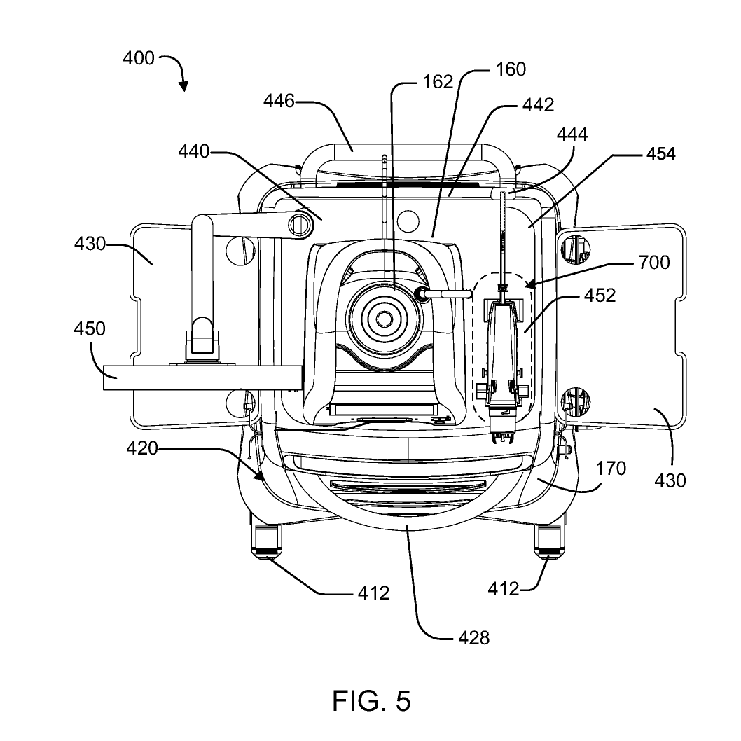

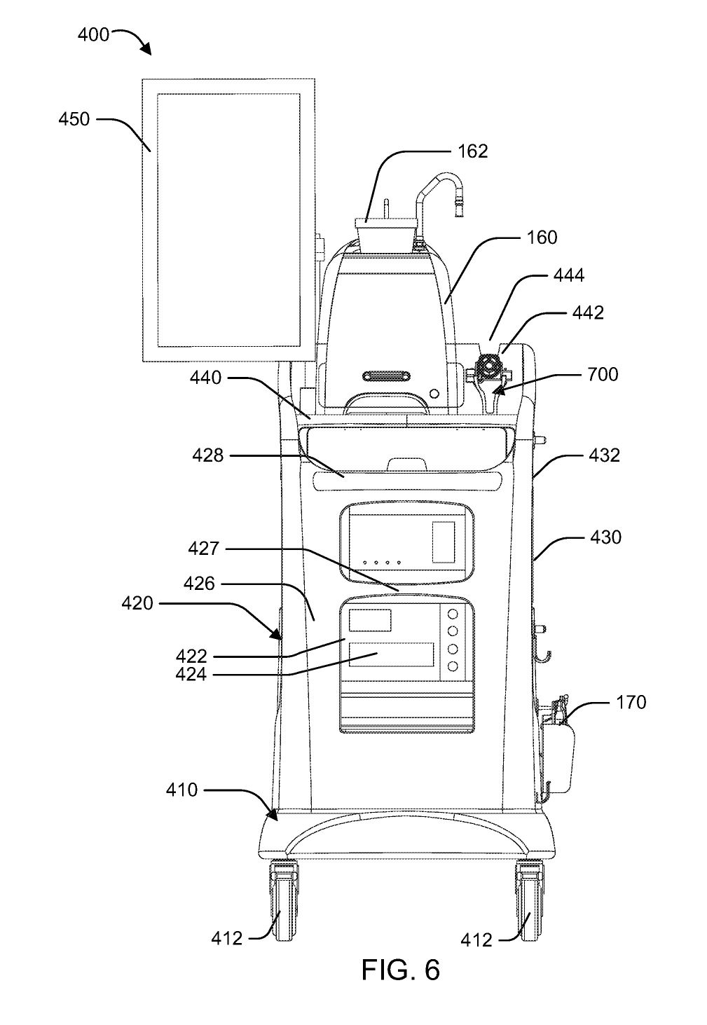

FIGS. 4-6 show an example cart 400 according to an aspect of the disclosure. FIG. 4 is a perspective view, FIG. 5 is a top view, and FIG. 6 is a front view of the cart 400. The cart 400 may include a base 410 that supports a cabinet 420. The base 410 may include wheels 412, which may be, for example, caster wheels. The cabinet 420 is configured store electronic equipment such as a specimen radiograph unit 422. Each side of the cabinet 420 includes collapsible shelves 430. Hooks 436 can be mounted to the side of the cabinet 420 for holding tubing 142,152 and wires 144, 154 (see FIG. 1). A front handle 428 extends from the front of the cabinet 420 and provide a primary grip for maneuvering the cart 400. A receptacle 438 can be mounted to the side of the cabinet 420 and retain the foot controls 170. The top surface 440 of the cabinet 420 is configured to support the control unit 160 and a biopsy device holder 700. The control unit 160 can support the collection canister 162. Alternatively, collection canister can be supported on the top surface 440 of cart 400. In the present example, a monitor 450 can be mounted on the cabinet 420, for example, via an adjustable arm 452. The biopsy device holder 700 is generally universal in character such that the device holder 700 is configured to hold medical devices of differing shapes and sizes. For example, the biopsy device holder 700 may be configured to hold vacuum assisted breast biopsy devices designed for use with either ultrasound or stereotactic breast biopsy procedures. In one embodiment, the biopsy device holder 700 remains in the upright position at all times. In another embodiment, the biopsy device holder 700 may be folded down into the top surface 440 of the cart 400 so that the cart has a smooth top surface during a medical procedure.

The biopsy device holder 700 includes one or more matched pairs of upwardly extending gripping arms. The upwardly extending gripping arms may either have completely flexible functionality or may have limited flexible functionality. The flexible functionality may be derived from having either flexible inserts affixed to non-flexible holding arms of the device holder 700 or by having completely flexible upwardly extending gripping arms configured in such a way as to supportively grip the medical device, while still allowing for rapid removal when the device is needed to be used.

The cabinet 420 may include a front panel 426 that may shield the electronic devices inside. For example, the cabinet 420 may store a specimen radiograph unit 422 such as, for example, the Mammotome® CoreVision unit.

The specimen radiograph unit 422 can be connected to the monitor 450. Although not shown, it should be understood that in some examples the cabinet 420 can also include an integrated medical grade isolation transformer to supply any suitable power to each electronic device such as specimen radiograph unit 422. In the present example, the specimen radiograph unit 422 includes a retractable sample tray 424. The sample tray 424, when in an open position, may extend through a cutout in the front panel 426 to allow access to the tray. For example, an operator may insert tissue samples into the sample tray 424 when in the open position. In an aspect, the cabinet 420 further includes a light source at the location 427. In examples using the light source, the light source is generally configured to illuminate the interior of sample tray 424. To facilitate illumination of the interior of sample tray 424, light source can be mounted in a variety of positions. For example, the light source may be mounted to a back side of the front panel 426 and coupled to the isolation transformer. The light source may be continuously on when the cart 400 is operational, or may be controlled by a switch. For example, the light source may power on only when the sample tray 424 is opened.

The shelves 430 are generally configured to be moveable between a horizontal position and a collapsed position. For example, in FIG. 4 and FIG. 5 the shelves 430 are shown in a horizontal position while in FIG. 6 the shelves 430 are shown in a collapsed position. A light source 432 may be provided in the cabinet 420 above each shelf 430. The light source 432 may be directed to illuminate the shelves 430 when in the horizontal position. In an aspect, the light source 432 may turn off when the shelf 430 is in the collapsed position. In an aspect, for example, a pressure switch 434 may be located in a recess of the cabinet 420 that retains the collapsed shelf 430. When the pressure switch 434 is pressed, the light source 432 may be turned off. In another aspect, the switch may be an optical switch, proximity switch, or other known switch responsive to the position of the shelf 430. The switch may be coupled to a circuit for controlling the light source 432.

The top surface 440 can be at least partially surrounded by a rail 442. For example, the rail 442 may extend upward from the top surface 440 and extend along the sides and back of the top surface 440. The rail 442 can include a notch 444. For example, the notch 444 can be aligned with the biopsy device holder 700 such that a needle (or protective sleeve) of the biopsy device may be positioned within the notch 444. Accordingly, the notch 444 can be configured to help retain the biopsy device while providing additional protection for the sharp needle. As best seen in FIG. 5, the cart 400 can also include a rear handle 446 extending from a rear side of the cabinet 420. The rear handle 446 can provide a convenient place to grasp the cart 400 from the rear side that may be spaced apart from the biopsy device holder 700. The top surface 440 can also include a recessed portion 454, which may be located beneath the biopsy device holder 700. The recessed portion 454 may retain liquids that may leak from the biopsy device, preventing such liquids from running onto a floor or into the electronic equipment. An absorbent cloth may be placed in the recessed portion to further retain liquids.

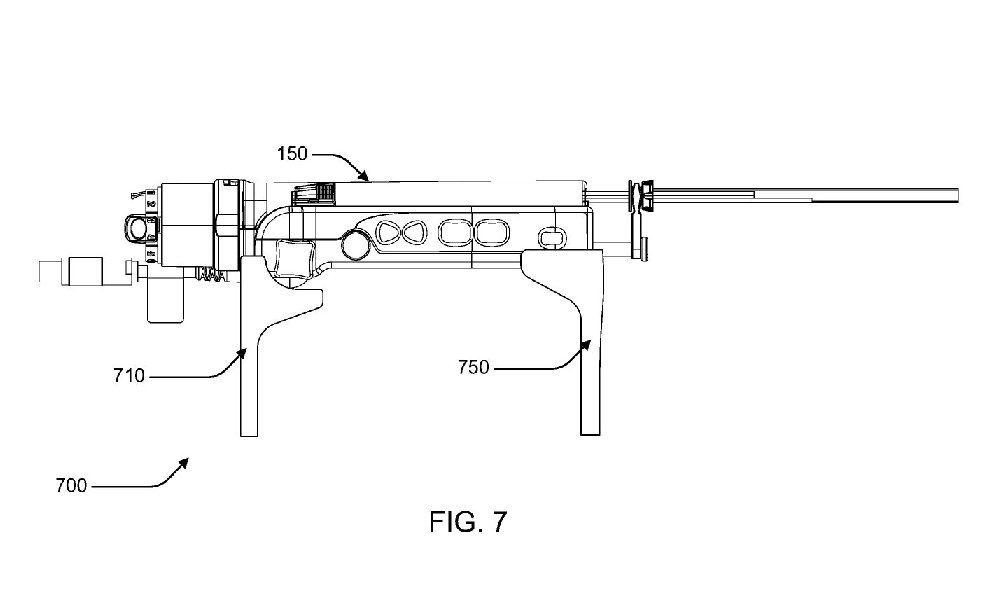

FIG. 7 is a side view illustrating an example biopsy device 150 and example biopsy device holder 700, according to an aspect of the disclosure. In an aspect, the device holder 700 may include a front cradle 710 and a rear cradle 750. The front cradle 710 may be mounted at the front of the cart 400 and may support a proximal end of the holster 310. The rear cradle 750 may be mounted at the rear of the cart 400 and may support a distal end of the holster 310. In an aspect, the device holder 700 may be a) mounted to a different location of the cart 400, b) may be mounted in a different orientation, or c) may be mounted to a different cart. Additionally, although the device holder 700 is illustrated as holding the biopsy device 150, the device holder 700 is can be characterized a generally universal device holder. Thus, the device holder 700 may be used to hold various biopsy devices known in the art. Further, in an aspect, the device holder 700 may be adapted to new biopsy device designs.

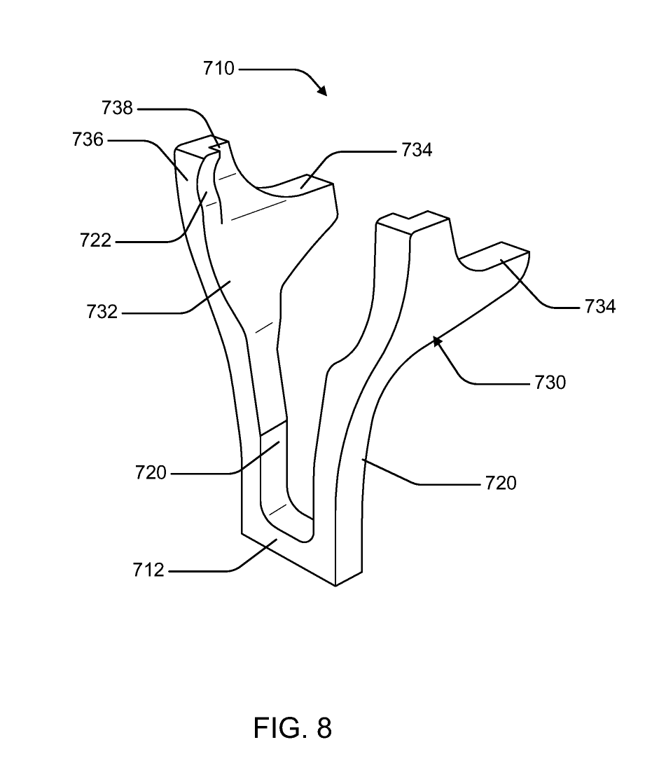

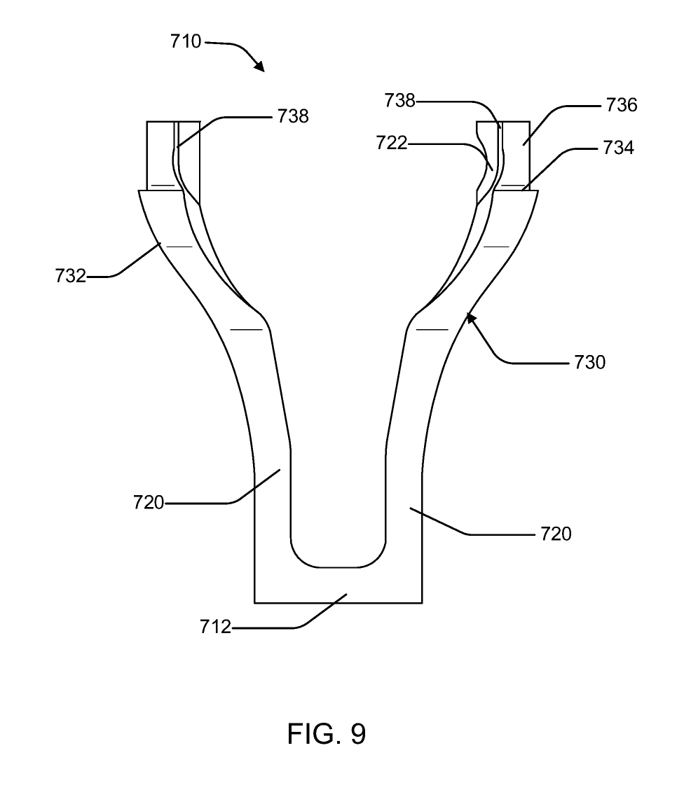

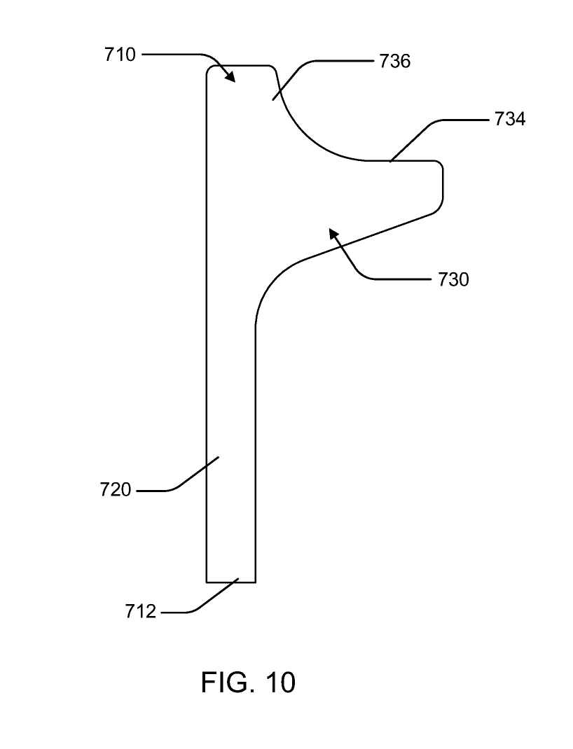

FIGS. 8-11 illustrate an example front cradle 710 of the device holder 700. FIG. 8 is a perspective view, FIG. 9 is a rear view, FIG. 10 is a side view, and FIG. 11 is a top view. The front cradle 710 may include a base 712 that may be mounted to the top surface 440. The base 712 may have a generally flat bottom surface. The base 712 can be mounted to the top surface 440 using one or more of fasteners, adhesive, welding, or other joining techniques known in the art. A pair of gripping arms 720 extend upwardly from the base 712. The arms 720 are generally be angled outward. The outward angle of arms generally decreases as arms 720 extend upwardly. For instance, the arms 720 are be nearly vertical near the base. The angle from vertical then decreases as the arms extend away from the base 712. For example, an angle between the top surface 440 and an outer surface of the arms 720 near the base 712 may be between 80° and 90° for a first portion. A second portion of the arms 720 may extend outward at a decreased angle. For example, an angle between the top surface 440 and an outer surface of the arms 720 in the second portion may be between 60° and 85°.

A portion of arms 720 includes a cradle portion 730. The cradle portion 730 defines a generally concave inner surface 732. In an aspect, the concave inner surface 732 may conform to a portion of the bottom surface of a biopsy device (e.g., biopsy device 140). In an aspect, the cradle portions 730 of the pair of arms 720 may form a partial cylinder. For example, each cradle portion 730 may form one side of a cylinder, and the cylinder may be open at the top and bottom between the two sides. The cradle portion 730 further extends rearward. Accordingly, inner surface 732 can contact the bottom surface of the biopsy device along a portion of the length of the bottom surface. For example, the cradle portion 730 can extend rearward between approximately 5 cm and 15 cm. A top surface 734 of the cradle portion 730 may be flat. In an aspect, a projection from the side of a biopsy device (e.g., stud 312) can rest on the top surface 734. A stop 736 can extend upward from the flat top surface 734. The stop 736 of each arm 720 may include an internal corner 738 that may face inwardly and rearward. In an aspect, a back edge of the biopsy device 150 may contact the stop 736 and fit within the internal corners 738. A notch 722 is generally located on an internal edge of the stop 736 for one or both arms 720. The notch 722 is generally configured to accommodate a feature of the biopsy device 150 such as a wire 154.

In an aspect, the front cradle 710 may be both flexible and resilient. For example, the arms 720 may deflect outward when a biopsy device is placed on the front cradle 710. In an aspect, each arm 720 may flex at least 10°, preferably at least 20°. The arms 720 may spring back toward their original position, applying a lateral force to the biopsy device 140, 150. In an aspect, the front cradle 710 may be formed from an elastomeric material. The front cradle 710 may be made from or coated with a slip resistant or gripping material. The example front cradle 710 may derive flexible functionality from completely flexible upward extending gripping arms 720.

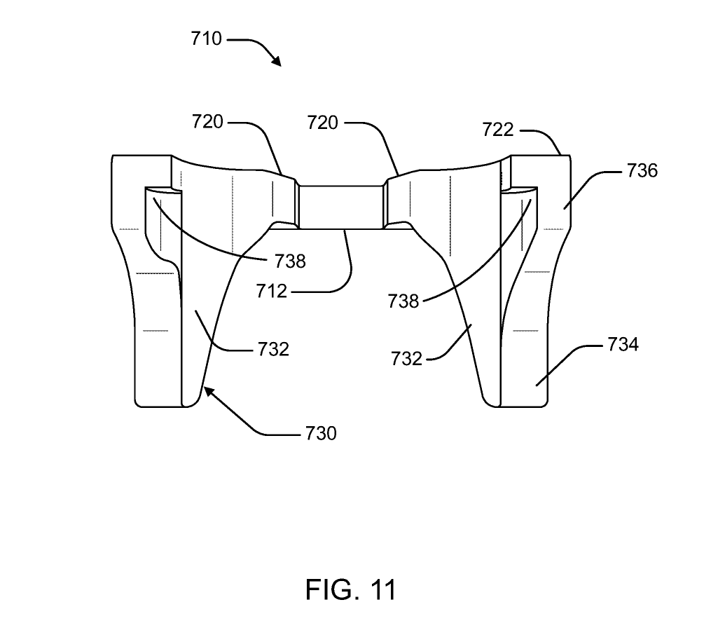

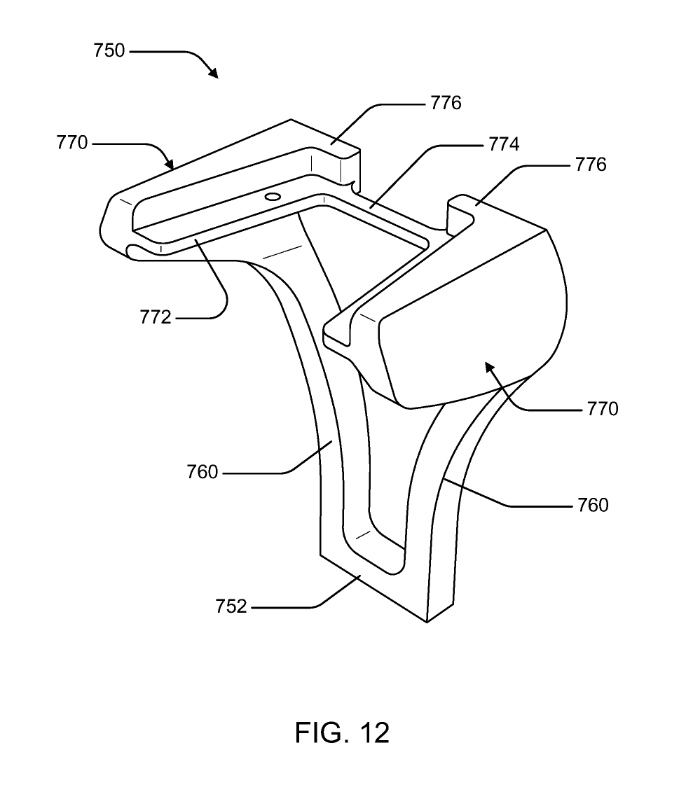

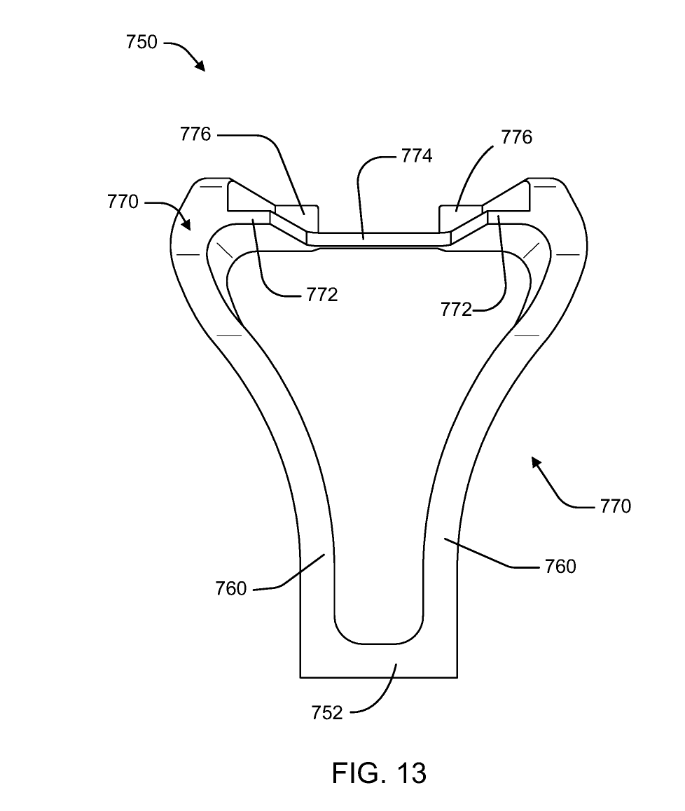

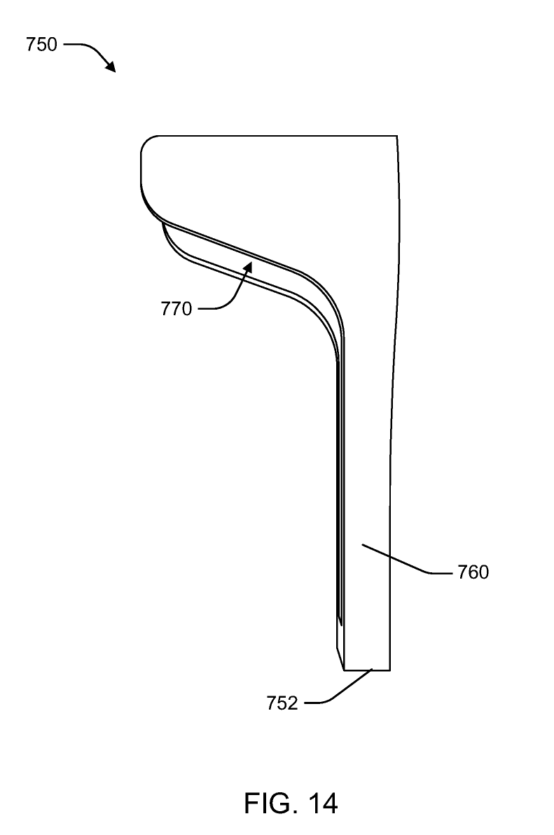

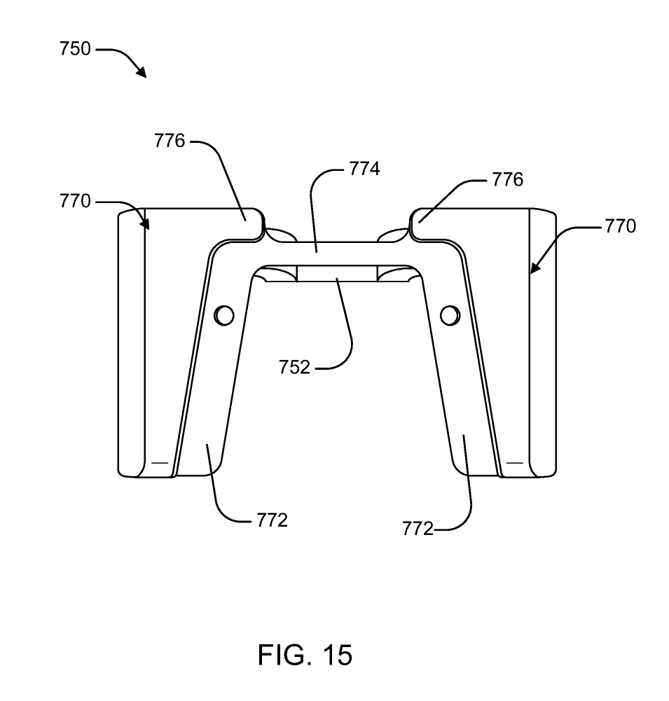

FIGS. 12-15 illustrate an example rear cradle 750 of the device holder 700. FIG. 12 is a perspective view, FIG. 13 is a front view, FIG. 14 is a side view, and FIG. 15 is a top view. The rear cradle 750 may include a base 752, arms 760, and a cradle portion 770. The base 752 may be mounted to the top surface 440. The base 752 includes a generally flat bottom surface. The base 752 can be mounted to the top surface 440 using one or more of fasteners, adhesive, welding, or other joining techniques known in the art. A pair of gripping arms 760 extend upwardly from the base 752. In some examples, the arms 760 can be generally matched with the arms 720, for example, as a set of front and rear arms. The arms 760 may generally be angled outward, and an angle from vertical may gradually decrease as the arms extend away from the base 752. In an aspect, the arms 760 may be nearly vertical near the base. For example, an angle between the top surface 440 and an outer surface of the arms 720 near the base 752 can be between 80° and 90° through a first portion. An angle between the top surface 440 and an outer surface of the arms 760 near the cradle portion 770 may be between 60° and 85° in a second portion.

Arms 760 generally define a cradle portion 770. The cradle portion 770 generally extends forward from the arms 760 toward the front cradle 710. For example, the illustrated rear cradle 750 may include curved outer surfaces for the cradle portion 770, but it should be appreciated that such features may be ornamental. In the present example, the cradle portion 770 generally extends forward between approximately 5 cm and 15 cm. An inner surface of the cradle portion 770 includes an inward extending flange 772. The flange 772 may be approximately 1 cm wide. The flange 772 may include a connecting portion 774 extending between the two cradle portions 770. The cradle portions 770 also includes a stop 776 extending above the flange 772. A biopsy device 140, 150 can be placed on the rear cradle 750 such that a distal end of the reusable holster 210, 310 may contact the stops 776. The flange 772 may support a bottom surface of the reusable holster 210, 310.

In an aspect, the rear cradle 750 may be both flexible and resilient. For example, the arms 760 can deflect outward when a biopsy device is placed on the rear cradle 750. In an aspect, however, the connecting portion 774 can limit the deflection of the arms 760. In another aspect, the cradle portion 770 can deflect outwardly while the arms remain relatively fixed. Accordingly, a distal end of the holster 310 may be supported on the connecting portion 774 while the cradle portion 770 may expand to accommodate a larger width of the holster in a proximal area. In an aspect, each cradle portion 770 may flex at least 10°, preferably at least 20°. The cradle portions 770 may spring back toward their original position, applying a lateral force to the biopsy device 140, 150. In an aspect, the rear cradle 750 may be formed from an elastomeric material. The rear cradle 750 may be made from or coated with a slip resistant or gripping material. The example rear cradle 750 may derive flexible functionality from completely flexible upward extending gripping arms 760.

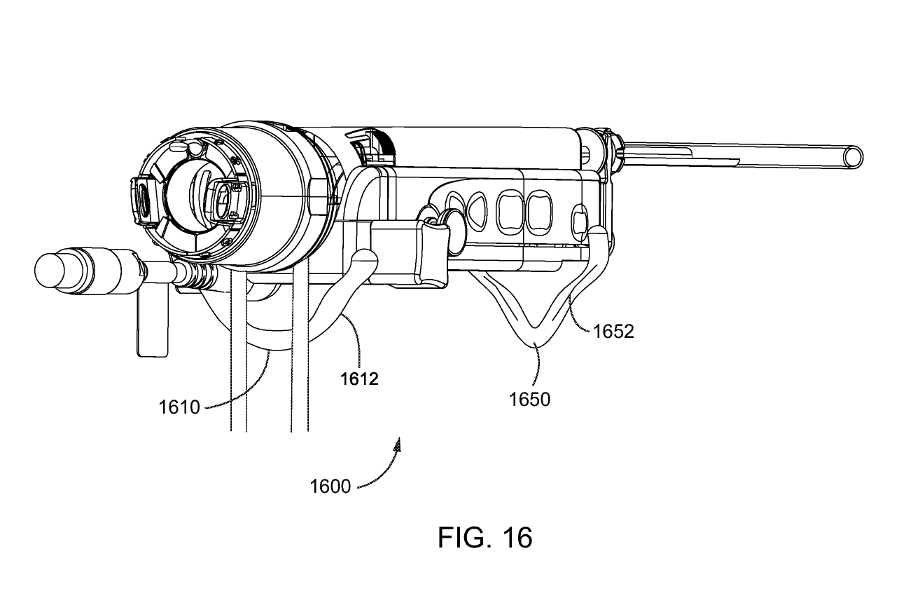

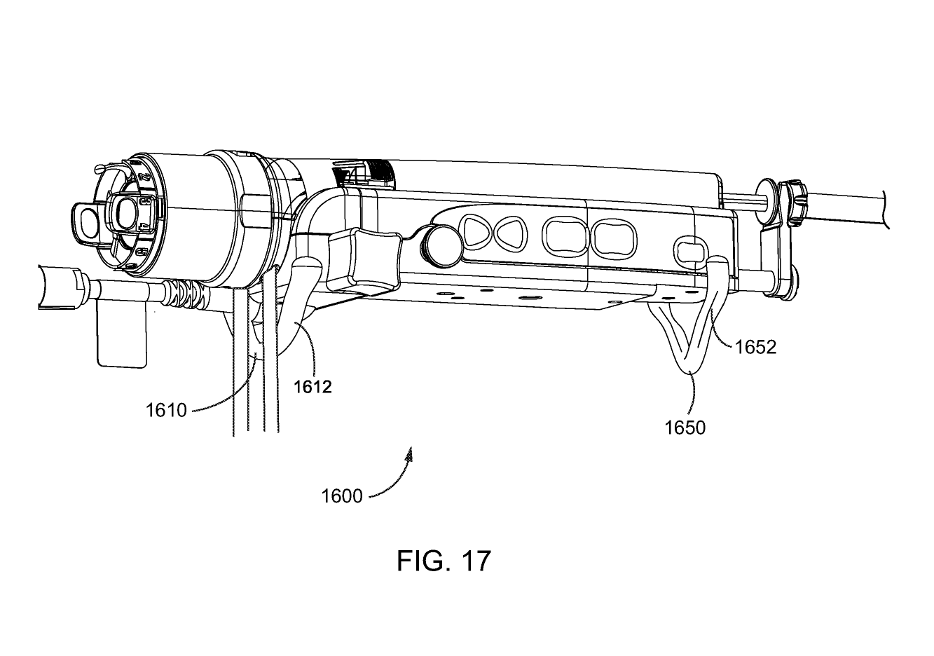

FIG. 16 is a perspective view showing another example biopsy device holder 1600 according to an aspect of the disclosure. FIG. 17 is another perspective view of the example biopsy device holder in FIG. 16. The biopsy device holder 1600 may include a front cradle 1610 and a rear cradle 1650. The front cradle 1610 includes a pair of upwardly extending arms 1612. In an aspect, the front cradle 1610 can be formed from a single reconfigurable element that may be bent or curved at the middle to distinguish the pair of arms. The front cradle 1610 can also include a mounting element at the middle. The mounting element may include, for example, a threaded tap through the reconfigurable element, a band surrounding the reconfigurable element, a clamp, or a hinge. The front cradle 1610 may be mounted directly to the top surface 440, or may be mounted on a support such as a vertical column. For example, the front cradle 1610 can be mounted in the same position as the front cradle 710.

The rear cradle 1650 similarly includes a pair of upwardly extending arms 1652. In an aspect, the rear cradle 1650 can be formed from a single reconfigurable element that may be bent or curved at the middle to distinguish the pair of arms 1652. In an aspect, the upwardly extending arms 1652 may be narrower than the upwardly extending arms 1612; in order to more tightly hold the distal portion of a biopsy device 140, 150. The rear cradle 1650 can also include a mounting element at or close to the middle. The mounting element may include, for example, a threaded tap through the reconfigurable element, a band surrounding the reconfigurable element, or a clamp. The rear cradle 1650 may be mounted directly to the top surface 440, or may be mounted on a support such as a vertical column. For example, the rear cradle 1650 may be mounted in the same position as the rear cradle 750.

In an aspect, the reconfigurable element can be bent to a particular shape and retain the particular shape. For example, the reconfigurable element may include a core made from a memory material and an elastomeric coating. The memory material may include, for example, nitinol, flexible plastic, or metal wire. In an aspect, the core may be a solid metal wire of sufficient thickness to support the biopsy device 140, 150. In another aspect, the core may be a bundle of wires. The wires may be woven, twisted, or wound to provide a desired degree of flexibility. The elastomeric coating may provide padding and increased friction where the reconfigurable element contacts the biopsy device 140, 150. For example, the elastomeric coating may include rubber, latex, or a soft plastic. The example front portion 1610 and rear cradle 1650 may derive flexible functionality from completely flexible upward extending gripping arms formed by the reconfigurable element.

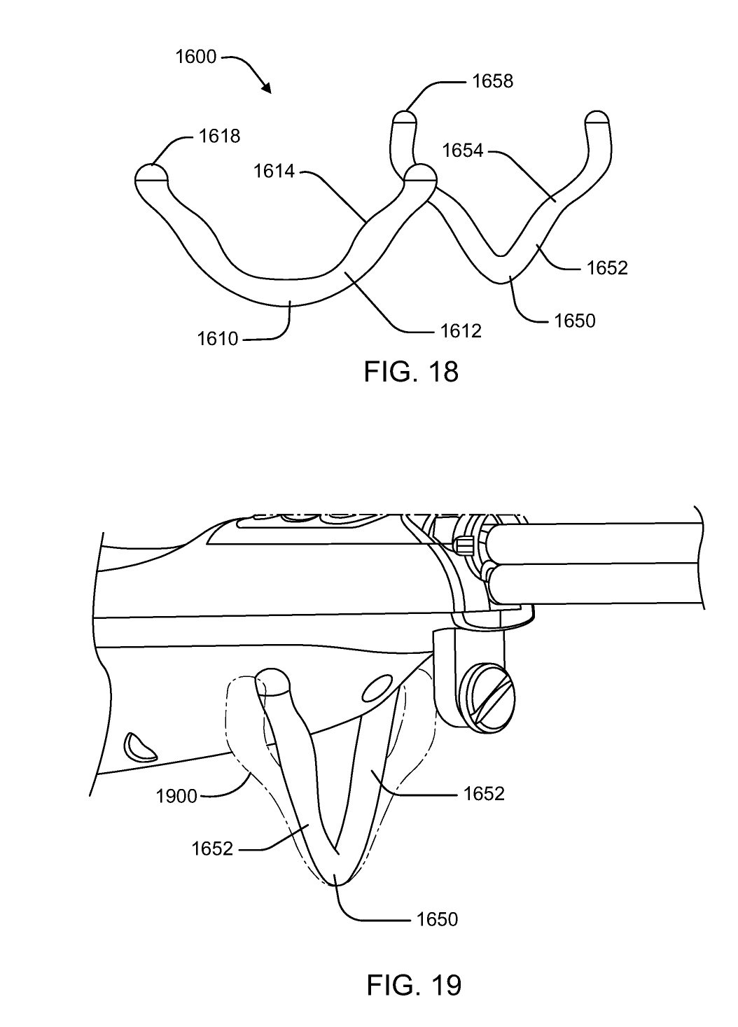

FIG. 18 is a perspective view showing the example biopsy device holder of FIG. 16 with the biopsy device not shown. As best seen in FIG. 18, the arms 1612 of the front cradle 1610 extend at approximately a 45° angle from the top surface 440. In other examples, the angle may be between about 30° and 60°. It should be appreciated, however, that the reconfigurable element may be bent to various angles. The arms 1612 may also include an inflection point 1614 separating a first portion of an arm 1612 from a second portion of the arm 1612. The two portions of the arm may conform to different portions of a biopsy device, or conform to different biopsy devices. The tips 1618 of the arms 1612 are generally rounded. In some examples, the tips 1618 may have a larger diameter than the portion of the arms 1612 to help retain the biopsy device.

The arms 1652 of the rear cradle 1650 generally extend upwardly at a steeper angle than the arms 1612. For example, the angle of the arms may be approximately 50°. In other examples, an angle of the arms 1612 may be between 35 and 75°. Because the arms may be reconfigurable, an angle may be selected based on the particular biopsy device to be supported. The arms 1652 also include an inflection point 1654 and tips 1658 similar to those of the front cradle 1610.

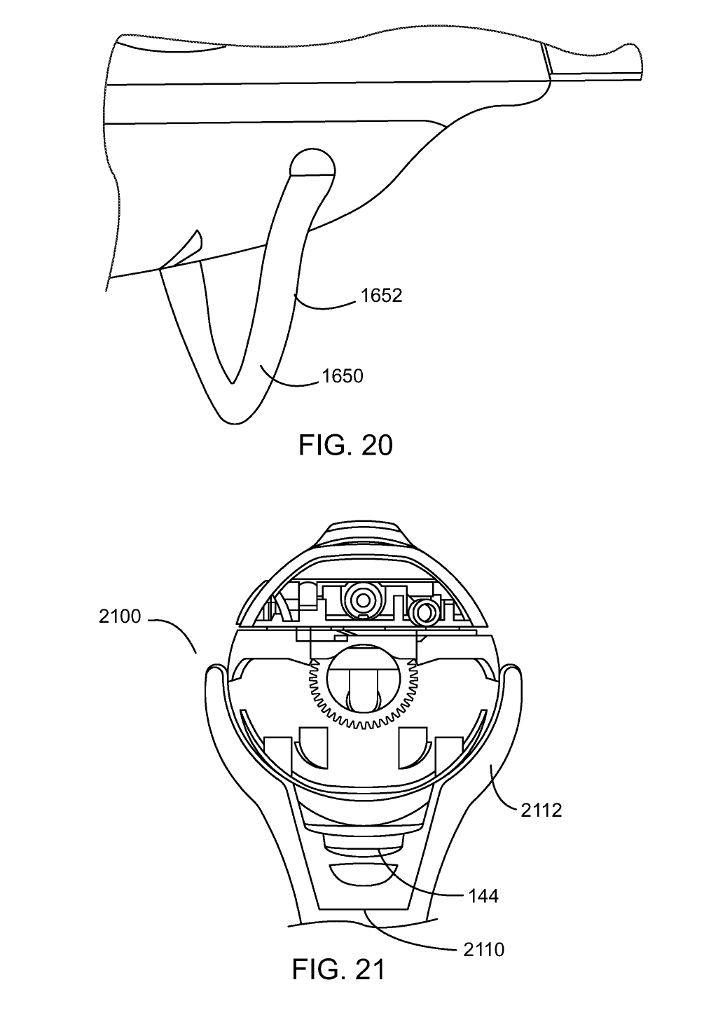

FIG. 19 is a perspective view illustrating reconfiguration of the example biopsy device holder 1600. The rear cradle 1650 may be used to support a distal portion of the holster 210 of the biopsy device 140. In an initial configuration (shown in dashed lines), the arms 1652 may be spread too wide. For example, the arms 1652 may have been configured for the biopsy device 150. In the initial configuration, the biopsy device may move within the arms 1652 or the distal end may sit lower than the proximal end of the device. As illustrated, the arms 1652 may be pinched in to better grip the biopsy device 140. The pinching may cause the core to change shape. For example, the pinching may change the angle of the arms 1652 by changing the shape at the middle of the rear cradle 1650. The arms 1652 may also be bent to conform the shape of the arms to the bottom surface of the biopsy device 140. Accordingly, the rear cradle 1650 may firmly grip the biopsy device 140, as illustrated in FIG. 20.

FIG. 21 is a front view of another example biopsy device and example biopsy device holder 2100 according to an aspect of the disclosure. The biopsy device holder 2100 may be similar to the biopsy device holder 1600. A front cradle 2110 of the biopsy device holder 2100 may support a proximal end of the biopsy device 140. The proximal end of the biopsy device 140 may include a rounded bottom surface and the wire 144 extending from the bottom surface. As illustrated, the arms 2112 may be bent to conform to the shape of the bottom surface. The space between the arms may provide space for the wire 144.

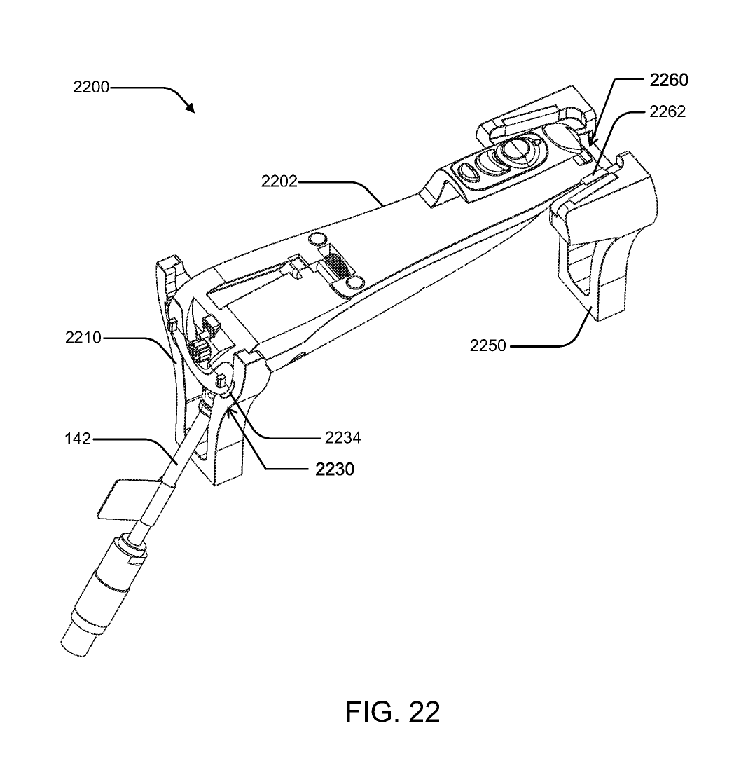

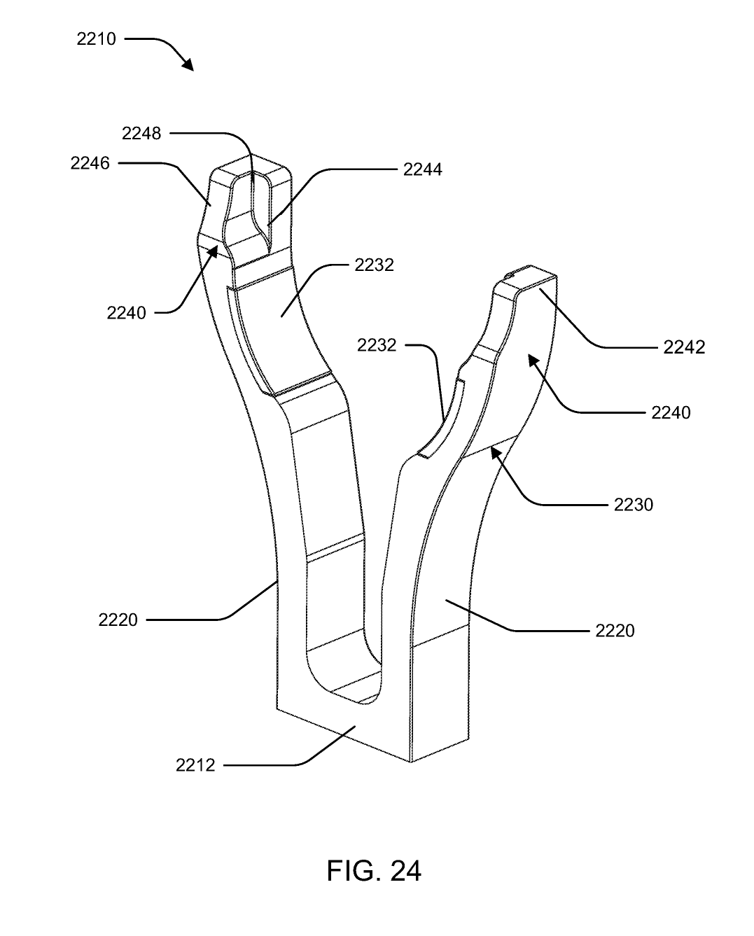

FIG. 22 is a perspective view illustrating an example device holder 2200 holding an example biopsy device 2202. The example biopsy device 2202 may be an ultrasound biopsy device similar to the biopsy device 140. Only the holster portion is shown to avoid obscuring the example biopsy device holder 2200. The device holder 2200 includes a front cradle 2210 and a rear cradle 2250. The biopsy device 2202 can be retained in a lower cradle portion 2230 of the front cradle 2210 and contact flexible inserts 2234, which grip a proximal portion the biopsy device 2202. The biopsy device 2202 can also be retained in a lower cradle portion 2260 of the rear cradle 2250 and contact flexible inserts 2262, which grip a distal portion of the biopsy device 2202. Further details of the device holder 2200 are discussed below with respect to FIGS. 24-32.

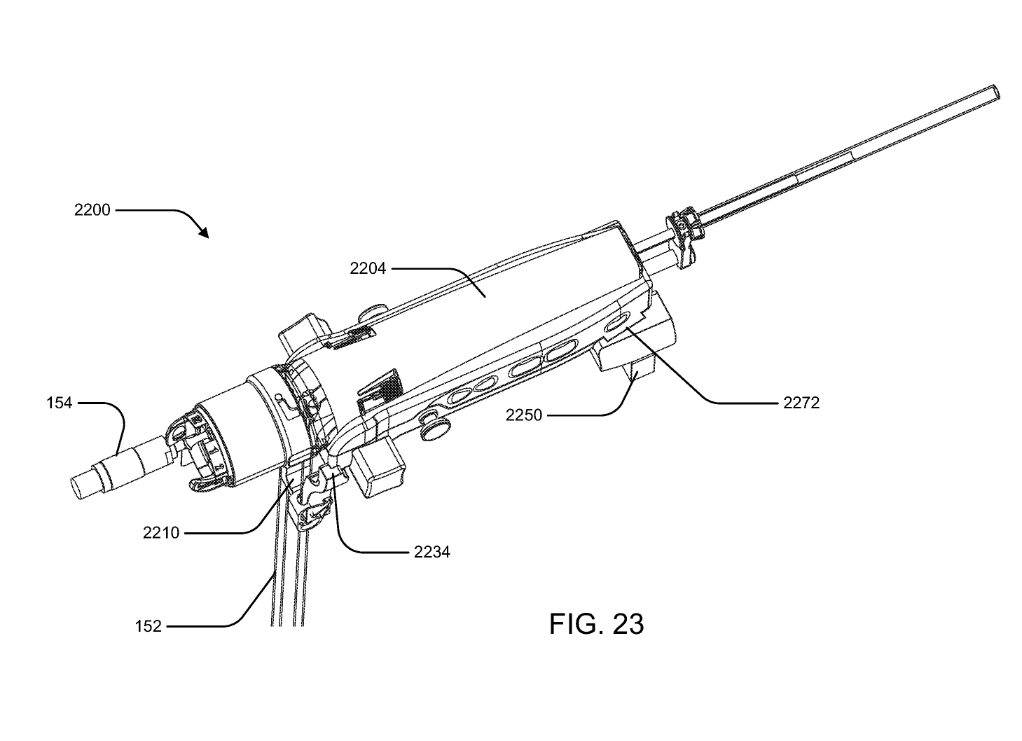

FIG. 23 is a perspective view illustrating the example device holder 2200 holding an example biopsy device 2204. The example biopsy device 2204 may be a stereotactic biopsy device similar to the biopsy device 500. The biopsy device 2204 can be retained in an upper cradle portion 2240 of the front cradle 2210. A stop 2242 conforms to a shape of a proximal end of the biopsy device 2204. The biopsy device 2204 can also be retained in an upper cradle portion 2270 of the rear cradle 2250 and contact flexible inserts 2272, which grip a distal portion of the biopsy device 2204. The gripping at the distal end of the biopsy device 2204 generally prevents movement of the sharp pointed end of the biopsy device, for example, if the tubing 152 or wires 154 are pulled.

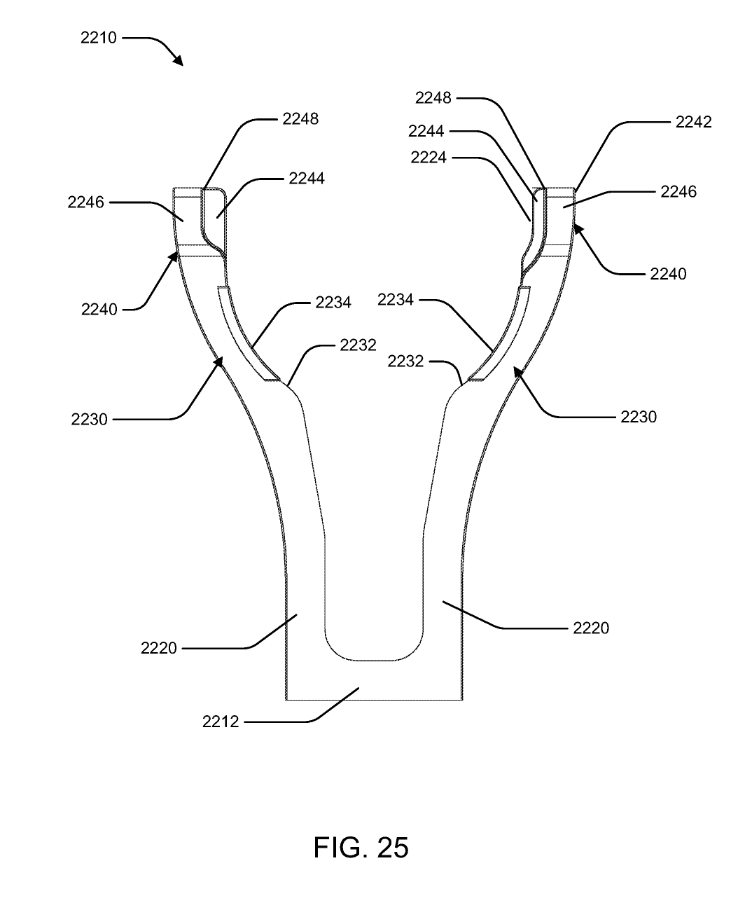

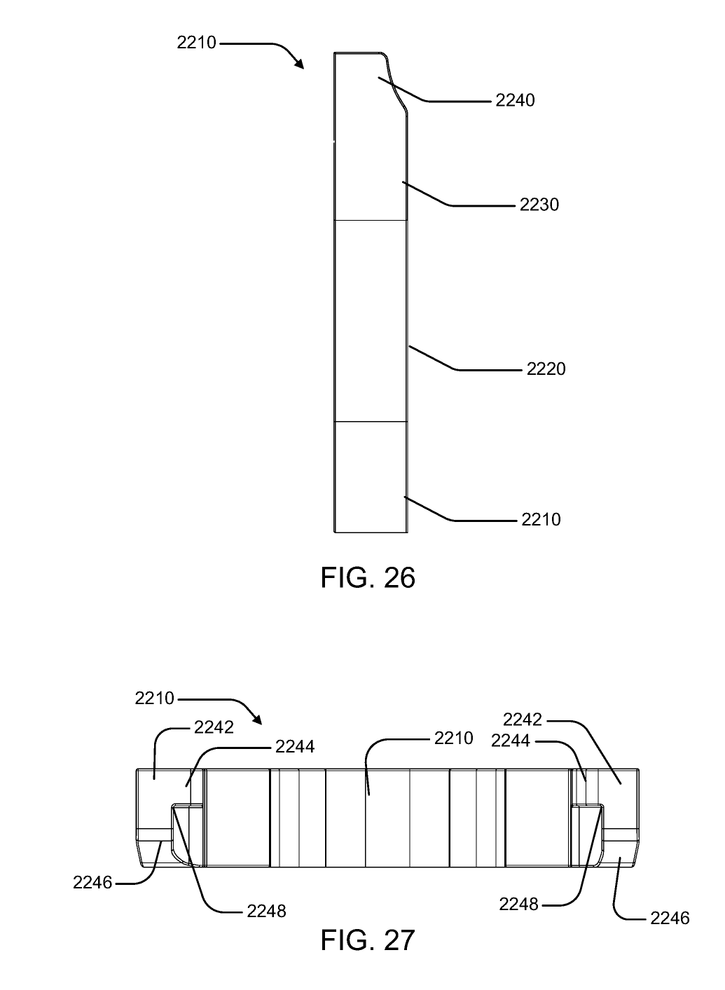

FIGS. 24-27 illustrate an example front cradle 2210 of the device holder 2200. FIG. 24 is a perspective view, FIG. 25 is a rear view, FIG. 26 is a side view, and FIG. 27 is a top view. The front cradle 2210 includes a base 2212 that can be mounted to the top surface 440. The base 2212 may have a generally flat bottom surface. The base 2212 can be mounted to the top surface 440 using one or more of fasteners, adhesive, welding, or other joining techniques known in the art. A pair of arms 2220 extend upwardly from the base 2212. The arms 2220 may generally be angled outwardly as the arms 2220 extend upwardly. In the present example, the arms 2220 are nearly oriented vertically near the base 2212. The angle from vertical may decrease as the arms extend upwardly from the base 2212. For example, an angle between the top surface 440 and an outer surface of the arms 2220 near the base 2212 may be between 80° and 90° for a first portion. A second portion of the arms 2220 may extend outward at a reduced angle. For example, an angle between the top surface 440 and an outer surface of the arms 2220 in the second portion can be between about 60° and 85°.

The arms 2220 include a lower cradle portion 2230 and an upper cradle portion 2240.

The lower cradle portion 2230 generally defines a concave inner surface 2232. As will be understood, the concave inner surface 2232 generally conforms to a portion of the bottom surface of a biopsy device (e.g., biopsy device 2202). The inner surface 2232 includes a flexible insert 2234 that contacts the bottom surface of the biopsy device near a proximal end of the biopsy device.

The upper cradle portion 2240 includes a stop 2242. The stop 2242 includes a front wall 2244 that contacts a proximal end of the biopsy device 2204 or a holster portion thereof. The stop 2242 of each upper cradle portion 2240 may also include a side wall 2246 connected to the front wall 2244 via an internal corner 2248 that may face inwardly and rearward. The front wall 2244, side wall 2246, and corner 2248 are generally configured to receive at least a portion of the biopsy device 2204. For instance, a proximal edge of the biopsy device 2204 may contact the stop 2242 and fit within the internal corners 2248. A notch 2224 may be located on an internal edge of the stop 2242 for one or both arms 2220. The notch 2224 is generally configured to accommodate one or more features of the biopsy device 2204 such as a wire 154.

In the present example, the front cradle 2210 is generally rigid. For example, the arms 2220 generally retain their shape and distance from each other. The flexible inserts 2234 generally provide flexibility for adapting to an inserted biopsy device 2202. The flexible inserts 2234 can also provide grip on a surface of the biopsy device 2202. By way of example only, the flexible inserts 2234 may be formed from an elastomeric material such as rubber to provide the flexible configuration of flexible inserts 2234. Thus, front cradle 2210 can generally derive flexible functionality from the flexible inserts 2234 affixed to non-flexible holding arms 2220.

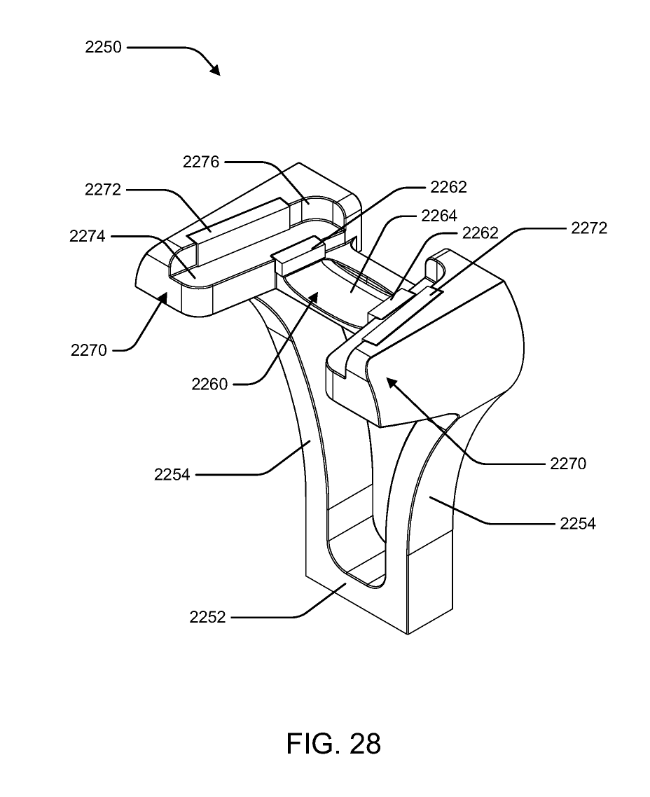

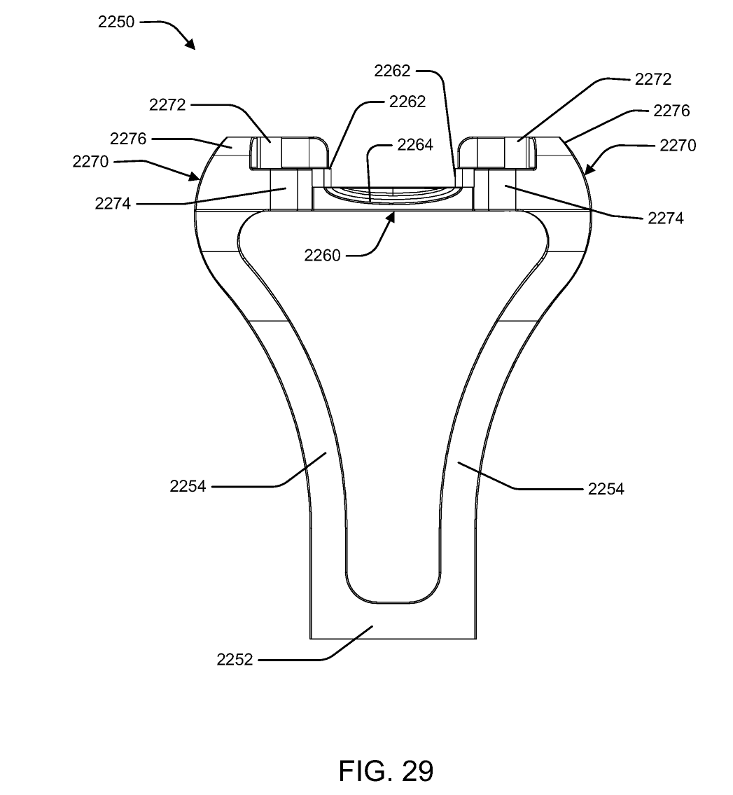

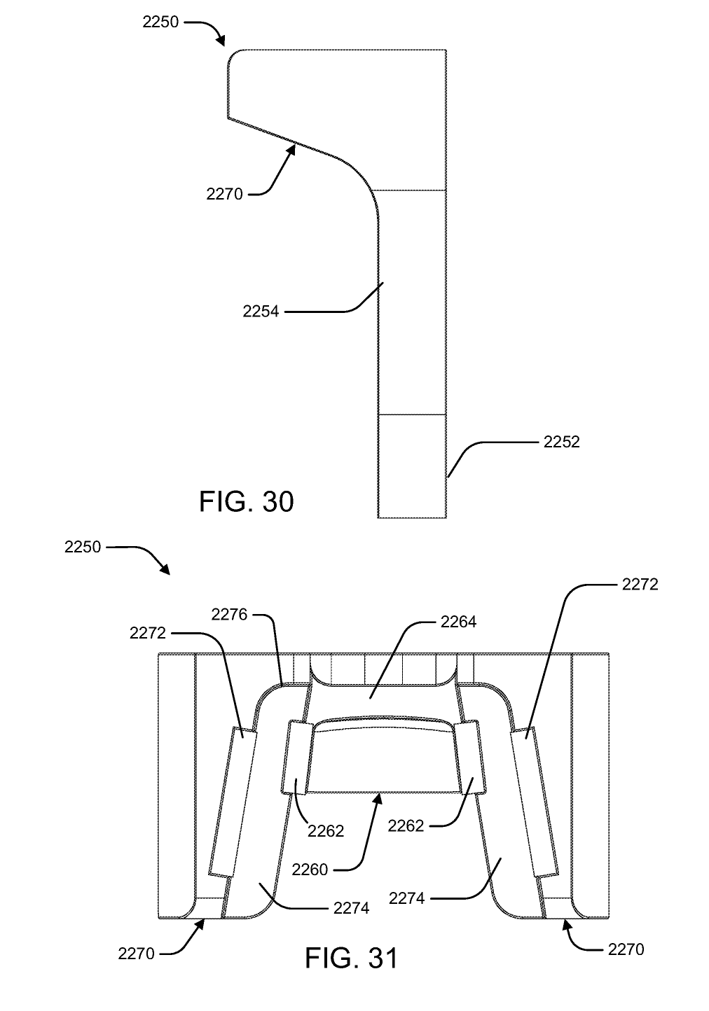

FIGS. 28-31 illustrate an example rear cradle 2250 of the device holder 2200. FIG. 28 is a perspective view, FIG. 29 is a front view, FIG. 30 is a side view, and FIG. 31 is a top view. The rear cradle 2250 includes a base 2252, arms 2254, a lower cradle portion 2260 and an upper cradle portion 2270. As described above, the base 2252 can be mounted to the top surface 440. The base 1152 may have a generally flat bottom surface. The base 2252 can be mounted to the top surface 440 using one or more of fasteners, adhesive, welding, or other joining techniques known in the art. A pair of arms 2254 extend upwardly from the base 2252. The arms 2254 are generally be angled outwardly, at an angle from vertical that gradually decreases as the arms extend away from the base 2252. The arms 2254 are generally configured to be nearly vertical near the base. For example, an angle between the top surface 440 and an outer surface of the arms 2254 near the base 2252 is generally between 80° and 90° for a first portion of extension. An angle between the top surface 440 and an outer surface of the arms 2254 near the lower cradle portion 2260 is generally between 60° and 85° for a second portion of extension.

The rear cradle 2250 includes a lower cradle portion 2260 and an upper cradle portion 2270. The lower cradle portion 2260 generally connects the arms 2254. For example, the lower cradle portion 2260 includes a bridge 2264 that spans between arms 2254. The bridge 2264 defines a generally concave top surface that conforms to a bottom surface of the biopsy device 2204. A distal end of the bottom surface of the holster portion the biopsy device 2202 may rest on the top surface of the bridge 2264. The lower cradle portion 2260 also includes flexible inserts 2262. The flexible inserts 2262 generally contact the curved sides of the biopsy device 2202. Thus, the flexible inserts 2262 can deform inward against pressure from the biopsy device 2202. The flexible inserts 2262 can also grip the curved surface of the biopsy device 2202. In an aspect, the gripping may prevent movement of the biopsy device 2202, for example, if the wire 144 is pulled downward.

The upper cradle portion 2270 extends forward from the arms 2254 toward the front cradle 2210. In the present example, rear cradle 2250 includes curved outer surfaces for the upper cradle portion 2270, but it should be appreciated that such features may be ornamental. The upper cradle portion 2270 can extend forward between approximately 5 cm and 15 cm. The inner surface of the cradle portion 2270 may include an inward extending flange 2274. The flange 2274 is approximately 1 cm wide. The upper cradle portion 2270 may also include a wall 2276. The wall 2276 may extend diagonally inward along a length of the flange 2274. The wall 2276 may curve inward to form a rear wall at a rear end of the flange 2274. Flexible inserts 2272 are generally located in the wall 2276. The biopsy device 2204 can be placed on the rear cradle 2250 such that a distal end of the biopsy device 2204 may rest on the flanges 2274. As a result, the flexible insert 2272 can deform against a side surface of the biopsy device 2204 and grip the side surface. In some examples, this gripping generally prevents movement of the biopsy device 2202, for example, if the tubing 152 or wire 154 is pulled downward.

In the present example, the rear cradle 2250 is generally rigid. In particular, the base 2252 and arms 2254 generally retain their shape and relative distance from each other. Accordingly, rear cradle 2250, except for the flexible inserts 2262 and 2272, is generally formed from a rigid material such as metal, wood, ceramic, or a rigid plastic. The flexible inserts 2262 and 2272, by contrast, can be formed from an elastomer such as rubber. Thus, rear cradle 2250 may derive flexible functionality from the flexible inserts 2262 and 2272 affixed to nonflexible holding arms 2220.

Various examples of biopsy device holders are described above. In some examples, one or more device holders (e.g., device holders 700, 1600, or 2200) may be repositionably attached to the cart 400. For example, any of the bases 712, 752, 2212, or 2252 may be dismounted from the cart 400. Another biopsy device holder may be mounted in place of the dismounted portion. Accordingly, the various front and rear cradles of the disclosed biopsy device holders may be interchanged. In other examples, one or more portions of a biopsy device holder can be mounted to the cart 400 using one or more hinges (not shown). The portion of the biopsy device holder may be pivoted downward to a horizontal portion. The top surface 440 may include a recess corresponding to the respective biopsy device holder. Accordingly, the hingedly mounted biopsy device holder portion can be folded down into the top surface 440 of the cart 400 so that the cart has a smooth top surface during the medical procedure. For example, the front cradles 710, 2210 may fold rearward and the rear cradles 750, 2250 may fold forward such that the respective flat surfaces of the biopsy device holder portions align with the top surface 440.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

By way of example only, embodiments described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta or gamma radiation, ethylene oxide, or steam.

Embodiments of the devices disclosed herein can be reconditioned for reuse after at least one use. Reconditioning may include any combination of the steps of disassembly of the device, followed by cleaning or replacement of particular pieces, and subsequent reassembly. In particular, embodiments of the devices disclosed herein may be disassembled, and any number of the particular pieces or parts of the devices may be selectively replaced or removed in any combination. Upon cleaning and/or replacement of particular parts, embodiments of the devices may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A biopsy system comprising:
(a) a cart for supporting a biopsy controller that controls biopsy of tissue; and
(b) a multi-device holder attached to the cart and including a holder support having a first cradle portion to receive and support a first biopsy device having a first exterior surface and a second cradle portion to receive and support a second biopsy device having a second exterior surface different from the first exterior surface.

2. The biopsy system of claim 1, wherein the holster support portion includes a stop configured to block movement of the first biopsy device or the second biopsy device relative to the support portion.

3. The biopsy system of claim 2, wherein the stop is defined by the first cradle portion of the holder support such that the stop is configured to engage the first biopsy device when the first biopsy device is supported by the first cradle portion.

4. The biopsy system of claim 2, wherein the stop includes a plurality of walls connected by an internal corner, wherein the walls are configured to engage the first biopsy device or the second biopsy device to prevent movement of the first biopsy device or the second biopsy device.

5. The biopsy system of claim 1, wherein the holder support includes a base and a pair of arms extending upwardly from the base, wherein the pair of arms is configured to support the first biopsy device or the second biopsy device away from a surface of the cart.

6. The biopsy system of claim 5, wherein each arm of each pair of arms extends upwardly while extending outwardly away from an opposite arm of the pair of arms.

7. The biopsy system of claim 6, wherein each arm of the pair of arms defines a first extension portion and a second extension portion, wherein the first extension portion of each arm defines an angle of extension between about 80° and 90°, wherein the second extension portion of each arm defines an angle of extension between about 60° and 85°.

8. The biopsy system of claim 7, wherein the first extension portion of each arm is adjacent to the base.

9. The biopsy system of claim 5, wherein each arm of the pair of arms is flexible such that each arm is configured to flex to receive the first biopsy device or the second biopsy device.

10. The biopsy system of claim 5, wherein each arm of the pair of arms is rigid such that each arm is configured to remain stationary in response to receipt of the first biopsy device or the second biopsy device.

11. The biopsy system of claim 10, wherein at least a portion of the holder support includes one or more flexible inserts, wherein each flexible insert of the one or more flexible inserts is configured to flex to receive the first biopsy device or the second biopsy device.

12. A biopsy system, the biopsy system comprising:
(a) a cart;
(b) a first biopsy holster defining a first exterior surface;
(c) a second biopsy holster defining a second exterior surface, wherein the second exterior surface is different from the first exterior surface; and
(d) a holder associated with the cart and defining a body and a pair of arms extending upwardly from the body, wherein each arm of the pair of arms defines an upper support portion and a lower support portion, wherein the upper support portion is configured to receive the first biopsy holster in lieu of receipt of the second biopsy holster in the lower support portion, wherein the lower support portion is configured to receive the second biopsy holster in lieu of receipt of the second biopsy holster in the upper support portion.

13. The biopsy system of claim 12, wherein the lower support portion includes a cradle portion extending between each arm of the pair of arms, wherein the cradle portion defines a curved surface corresponding to at least a portion of the second biopsy holster.

14. The biopsy system of claim 13, wherein the lower support portion further includes a pair of flexible inserts disposed adjacent to the cradle portion on opposite sides of the cradle portion.

15. The biopsy system of claim 14, wherein each flexible insert is configured to flex in response to receipt of the second biopsy holster within the cradle portion.

16. The biopsy system of claim 14, wherein each flexible insert is configured to grip at least a portion of the second biopsy holster upon receipt of the second biopsy holster within the cradle portion.

17. The biopsy system of claim 12, wherein the body of the holder is secured to an upper surface of the cart by a coupling, wherein the coupling is configured to permit folding of the holder to provide a work surface when the hold is not in use.

18. The biopsy system of claim 12, wherein the cart includes one or more foldable trays, wherein the one or more foldable trays are configured to fold to a horizontal position from at least one side of the cart.

19. The biopsy system of claim 18, wherein the cart further includes a light source and a pressure sensitive switch, wherein the pressure sensitive switch is configured to respond to the one or more foldable trays to activate the light source thereby illuminating a surface of the one or more foldable trays.

20. A biopsy system comprising:
(a) a cart defining an outer surface; and
(b) a cradle including a base coupled to the outer surface of the cart and a pair of arms extending upwardly from the base, wherein each arm of the pair of arms defines an upper support and a lower support, wherein the upper support is configured to support a first biopsy device holster, and the lower support is configured to support a second biopsy device holster in lieu of the first biopsy device holster being disposed within the upper support.
